(19) United States
(12) Patent Application Publication
(10) Pub. No.: US 2019/0224090 A1
(43) Pub. Date: Jul. 25, 2019

(54) NAIL POLISH FORMULATION

(71) Applicant: Yoram Fishman, Riverside, CA (US)

(72) Inventor: Yoram Fishman, Riverside, CA (US)

(21) Appl. No.: 15/875,290

(22) Filed: Jan. 19, 2018

(51) Int. Cl. A61K 8/34; A61Q 3/02; A61K 8/368; A61K 8/42; A61K 8/73; A61K 8/81

(52) U.S. Cl. CPC A61K 8/34; A61Q 3/02; A61K 8/368; A61K 2800/43; A61K 8/731; A61K 8/8158; A61K 8/342; A61K 8/42

(57) ABSTRACT

A nail polish composition includes an alcohol solvent; a cellulose polymer material; an acrylic co-polymer material; isostearyl alcohol; a plasticizer; and a colorant. The nail polish composition may be applied to the nails of a human or other animal, and may be removed with an alcohol material, without the use of toxic cleaning chemicals such as acetone.

### BACKGROUND OF THE INVENTION

### Field of the Invention

This invention relates generally to nail polish, and more particularly to a nail polish that may be removed with alcohol.

### Description of Related Art

Modern nail polish typically includes a film-forming polymer such as nitrocellulose dissolved in a volatile organic solvent such as butyl acetate. A concern for many has been that these chemicals, and also toxic chemicals which are used to remove the nail polish, can be harmful to users, and to professionals who apply the nail polish.

There is a long-felt need in the industry for a nail polish composition that avoids these health concerns by using a composition that includes an alcohol solvent. The present invention fulfills these needs and provides further advantages a s described in the following summary.

### SUMMARY OF THE INVENTION

The present invention teaches certain benefits in construction and use which give rise to the objectives described below.

The present invention provides a composition suitable for application as a nail polish, the composition comprising: an alcohol solvent; a cellulose polymer material; an acrylic co-polymer material; isostearyl alcohol; a plasticizer; and a colorant.

A primary objective of the present invention is to provide a nail polish composition having advantages not taught by the prior art.

Another objective is to provide a nail polish composition that does not include harsh and toxic chemicals which are harmful to a human or other animal that has the composition applied to its nails.

Another objective is to provide a nail polish composition that may be removed with alcohol, without the use of harsh and toxic chemicals.

A further objective is to provide a nail polish composition that does not have an offensive chemical smell, but which may include a fragrance so that the composition has an attractive smell.

Other features and advantages of the present invention will become apparent from the following more detailed description which illustrate, by way of example, the principles of the invention.

### DETAILED DESCRIPTION OF THE INVENTION

The above-described drawing figures illustrate the invention, a composition suitable for application as a nail polish. The composition comprises ingredients that are not harmful to the person's nails and health, and furthermore the composition may be removed with alcohol rather than more toxic chemicals typically used.

The composition includes an alcohol solvent. By providing the composition with an alcohol solvent as its base, the nail polish may be removed with alcohol rather than acetone or other powerful and damaging chemical. In this embodiment, the alcohol includes ethyl alcohol, and includes effective amounts of denaturing ingredients, for consumer safety. For purposes of this application, the term “effective amount” is defined to mean an amount deemed suitable by one skilled in the art for achieving the desired goal, in this case to prevent consumption of the alcohol. In the present embodiment, the specially denatured (SD) ethyl alcohol is specially denatured alcohol (SDA) 40-B 200 Proof. This includes denatonium benzoate as the denaturing ingredient. In this embodiment, to every 100 gallons of ethyl alcohol, 1/16 avoirdupois ounce of denatonium benzoate, NF, is added, along with ⅛ gallon of tert-butyl alcohol.

The composition includes a cellulose polymer material and an acrylic co-polymer material, both of which are soluble in alcohol. In this embodiment, the cellulose polymer material is an ethylcellulose polymer material, such as ETHOCEL®, which is sold by Dow Chemical. In another embodiment, the cellulose polymer material is hydroxypropyl cellulose, or hydroxyethyl cellulose.

In this embodiment, the acrylic co-polymer material is an acrylates/octylamide copolymer that includes a hydrophobic, high molecular weight carboxylated acrylic copolymer that is a film forming agent that is moisture resistant, and resistant to abrasion. In one embodiment, the acrylic co-polymer material is DERMACRYL® 79, sold by AkzoNobel Surface Chemistry. In another embodiment, the acrylic co-polymer material includes another form of octylacrylamide/acrylates/butylaminoethyl methacrylate copolymer.

The composition further includes isostearyl alcohol, a plasticizer, and a colorant. The isostearyl alcohol and the plasticizer are carefully selected and provided in an amount that results in the composition being stable, having a viscosity that is suitable for application as a nail polish, and that will dry in less than 5 minutes. In this embodiment, the plasticizer is a citric acid ester such as CIRTOFLEX®2, which is sold by Vertellus Corporation. In another embodiment, the plasticizer includes Tributyl Citrate, Acetyl Triethyl Citrate, Acetyl Tributyl Citrate, Acetyl Trihexyl Citrate, or an equivalent material.

Prior to use, the composition should be liquid that is thin and viscous enough to be easily be spread on the nails of a person in a thickness that is deemed appropriate to consumers and to those skilled in the art, while also having a fast enough drying time to be deemed acceptable to consumers and to those skilled in the art. The composition is deemed “dry” when it is no longer damaged or transferred to other surfaces through gentle contact.

Extensive experimentation has determined a composition suitable for application as a nail polish, the composition comprising:
SD alcohol 50.0-95.0% by weight;
ethocellulose polymer material 1.0-5.0% by weight;
an acrylic co-polymer material 5.0-25.0% by weight;
isostearyl alcohol 2.0-8.0% by weight;
a plasticizer 0.25-10.0% by weight; and
a colorant.

In one embodiment, the composition includes the following (all percentages by weight):
SD alcohol 72.55%;
Ethocel 2.0%;
Dermacryl 15.0%;
isostearyl alcohol 4.0%;
Citroflex2 0.25%;
Dow Corning CB 3046 Fluid 1.0%;
Permethyl 99A 2.0%;
Colorant 1.2%; and
Fragrance 2.0%.

In this embodiment, the composition further includes about 2% of an alcohol-soluble fragrance, such as an alcohol-soluble fragrance oil, or other suitable fragrance known in the art. In one embodiment, the fragrance is an oil sold by Gorgio®, type CP00264. Obviously, one skilled in the art may adjust the quantities of these ingredients, and he or she may substitute the ingredients with equivalent materials, according to the practices of those skilled in the art, and these equivalent formulations should be considered within the scope of the present invention.

As used in this application, the words “a,” “an,” and “one” are defined to include one or more of the referenced item unless specifically stated otherwise. The terms “approximately” and “about” are defined to mean+/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by the following claims.

1. A composition suitable for application as a nail polish, the composition comprising:
an alcohol solvent;
a cellulose polymer material;
an acrylic co-polymer material in an amount of 11-25% of the composition, by weight;
isostearyl alcohol;
a plasticizer comprising citric acid esters in an amount of 0.25-2.0% of the composition, by weight; and
a colorant, the composition being in liquid form.

2. The composition of claim 1, wherein the alcohol solvent is specially denatured, including a denaturing ingredient.

3. The composition of claim 2, wherein the specially denatured alcohol includes effective amounts of denatonium benzoate and tert-butyl alcohol.

4. The composition of claim 1, wherein the cellulose polymer material is an ethylcellulose polymer material.

5. The composition of claim 1, wherein the acrylic co-polymer material is an acrylates/octylamide copolymer.

6. The composition of claim 1, further comprising a fragrance.

7. The composition of claim 6, wherein the fragrance is an alcohol-soluble fragrance.

8. The composition of claim 7 wherein the alcohol-soluble fragrance is about 2% of the composition, by weight.

9. A composition suitable for application as a nail polish, the composition comprising:
alcohol 50.0-95.0% by weight;
ethocellulose polymer material 1.0-5.0% by weight;
an acrylic co-polymer material 11-25.0% by weight;
isostearyl alcohol 2.0-8.0% by weight;
a plasticizer comprising citric acid esters in an amount of 0.25-10.0% of the composition, by weight; and
a colorant, the composition being in liquid form.

10. The composition of claim 9, further comprising a fragrance.

11. The composition of claim 10, wherein the fragrance is an alcohol-soluble fragrance.

12. The composition of claim 11, wherein the alcohol-soluble fragrance is about 2% of the composition, by weight.

13. A method for coloring a nail, the method comprising the steps of:
providing a nail polish composition in liquid form, the nail polish composition comprising:
SD alcohol 50.0-95.0% by weight;
ethocellulose polymer material 1.0-5.0% by weight;
an acrylic co-polymer material 11-25.0% by weight;
isostearyl alcohol 2.0-8.0% by weight;
a plasticizer comprising citric acid esters in an amount of 0.25-10.0% of the composition, by weight; and
a colorant; and
applying the nail polish composition to the nail.
